(19) United States
(12) Patent Application Publication
(10) Pub. No.: US 2010/0249488 A1
(43) Pub. Date: Sep. 30, 2010

(54) METHOD OF CONTACTLESS MAGNETIC ELECTROPORATION

(75) Inventors: Thomas Joseph Kardos, Aliso Viejo, CA (US); Dietmar Paul Rabussay, Solana Beach, CA (US)

Correspondence Address: Thomas Joseph Kardos, 23531 Lipari, Laguna Hills, CA, 92653, US

(73) Assignee: MagneGene, Inc., Laguna Hills, CA (US)

(21) Appl. No.: 12/732,195

(22) Filed: Mar. 26, 2010

Related U.S. Application Data
(60) Provisional application No. 61/164,471, filed on Mar. 30, 2009.

(51) Int. Cl. A61N 2/04; A61N 2/00

(52) U.S. Cl. 600/14; 600/9

(57) ABSTRACT

This invention provides a novel method of tissue electroporation that eliminates the need for electrodes that conduct electricity to the tissues. This invention creates electric currents and fields sufficient for porating cell membranes for improving the delivery of polynucleotides such as plasmid and linear DNA and RNA constructs, and polypeptides such as antigen protein constructs into mammalian eucaryotic cells purely by magnetic field pulses that does not require the use of contacting electrodes to conduct electric or ionic current. This invention thus provides a method for improving transfection and immunogenicity of pharmaceutical substances without direct contact with a living body, and may be called magnetopermeabilization. A concomitant aspect of the invention is the method by which a drug such as a solution containing DNA is delivered to a targeted tissue bed that is optimal in conjunction with magnetopermeabilization for maximal transgene expression and drug effect.

This application claims priority to Provisional Application 61/164,471 filed Mar. 30, 2009.
Assignment: MagneGene, Inc., a California Corporation.

### FIELD OF THE INVENTION

The present invention relates to the method of delivery of therapeutic substances including polynucleotides and polypeptides into mammalian eukaryotic cells by inducing permeabilization of cell membranes with magnetic pulses. Particularly, the present invention relates to permeabilizing tissues near the surface of the body such as dermis, epidermis, sub-dermal regions, muscle tissues and tumor tissues by magnetically inducing electroporation without placement of electrodes onto, or within these tissues that conduct electricity to said tissues. Therefore, the present invention provides for a novel method for electroporation of cells that does not require physical contact with said tissues and thus does not require sterile or non-sterile electrodes to deliver energy. This method is related to the principle of magnetic induction of electrical currents and fields for the purpose of causing electroporation or electropermeabilization of cell membranes, and may be called magnetopermeabilization. Another aspect of the invention relates to methods of delivering agents, including drugs, to target tissues that work optimally with magnetopermeabilization.

### BACKGROUND OF THE INVENTION

The technique of electroporation generally involves the positioning of an electric field of a certain strength and direction across a suspension of cells or a segment of tissue whereby the portion of the field across each cell membrane is generally sufficient to rearrange the structure of that membrane to create temporary pores in the lipid bilayer. Provided the strength and direction of the applied field is within an appropriate range, the induced porosity is temporary and spontaneously re-seals after the energy that created the electric field is removed. While the pores are open, the cell membrane is permeable to fluids and dissolved ions and molecules, including macromolecules such as polynucleotides and polypeptides. Pulses of high field strength and long duration generally will induce a large quantity and/or pores of large size that may lead to apoptosis or cell lysis. Conversely, pulses of low field strength and/or short duration may induce an insufficient quantity or size of pores that will allow only a low flux rate of ions and molecules across the cell membrane.

The range of electric fields used for electroporation is in the tens to thousands of volts per centimeter, and has heretofore been generated in a variety of ways with two or more electrodes in various configurations, depending on the cells to be electroporated and the environment surrounding the cells. For example, cells in suspension are usually electroporated in chambers with electrodes of opposite polarity on opposite sides of the chamber. Cells in living tissue can be electroporated with either non-invasive electrodes in contact with the tissue surface (e.g., plate electrodes), or with invasive electrodes which penetrate into the tissue (e.g., needle electrodes). A variety of needle electrode configurations have been developed, encompassing, for example, two (Elgen 1000, Inovio), three (Tri-Grid, Ichor Medical Systems), four (MedPulser, Genetronics), five (Cellectra, VGX Pharmaceuticals) or six (MedPulser EPT, Genetronics) electrodes arrayed in various geometries. Such tissue penetrating electrode arrays are disclosed, for example, in U.S. Pat. Nos. 6,041,252, 6,278,895, and 7,245,963. Single needle electrodes have also been used, such as disclosed in U.S. Pat. No. 6,654,636 by Rabussay and others, or U.S. App 61/011,772 (2008) and PCT/US2009/000273 by Kardos and others. Large arrays of electrodes or microneedles have also been applied, such as eight or more electrodes (Derma Vax, Cyto Pulse), as disclosed in U.S. Pat. No. 6,119,660. What is common to all of these techniques is that they use metal electrodes that are generally in contact with the skin and penetrate to some degree into the tissue to be electroporated. Penetrating electrodes tend to use sharp needles that break through the stratum corneum barrier and into or through the dermis, which causes trauma, pain and risk of infection. Such tissue-piercing needle electrodes also require a complex manufacturing process governed by medical device manufacturing regulations and further involve sterilization and the need to maintain sterile packaging, thereby causing a significant cost per patient use.

To overcome the disadvantages of the use of non-invasive or invasive electrodes, attempts at needleless approaches have been described, such as using a corona discharge method disclosed in U.S. Pat. No. 6,929,949 (University of South Florida). This approach uses extra long duration pulses to rain electric-charge-carrying ions and radicals through an air gap onto the tissue. Each time a charge-carrying entity comes into contact with the skin, its charge is transferred, and over time, this results in a current or electric field within the tissue. Since the resultant current is very low due to the sparseness or low density of charge-carrying entities in the air as compared to an aqueous solution, it is necessary to maintain the corona effect for many minutes or hours to accumulate the equivalent electrical energy delivered by traditional electroporative pulses of micro to millisecond duration, or high voltage pseudo-spark type pulses of nanosecond duration. The effectiveness of the corona effect also depends on the ability to create a sufficient ionic wind or mass flow of ions in the air focused onto a particular target location. High voltage nano-second pulses with pseudo-spark devices to effect cells and ions in tissue have been used, such as described in U.S. Pat. No. 6,326,177 and in US App 2003/0170898 and in other issued patents and publications. Since the electrical charge and energy delivered is related to both the strength of the electrical field as well as its duration, sufficient energy to cause electroporation can be delivered by short nano-second pulses of high voltages (over 10 kilovolts). The capacitive effect of cell membranes within live tissues that contain electrolytic fluids and ions will also naturally lengthen the effective pulse duration of brief high voltage pulses, which may contribute to the completion of pore formation before the impulse energies completely dissipate.

Another demonstrated method of porating tissues and cell membranes is through a mechanical process as employed by the gene gun disclosed in U.S. Pat. No. 6,436,709. This approach uses nano-meter size gold or other particles coated with a desired macromolecule and shoots them under pressure from a gas-filled canister into the skin at selectable depths. This technique has been successful in delivering macromolecules past cell membranes into the cytoplasm, but also requires a complex manufacturing process to successfully apply the macromolecules onto the gold beads or other particles in sufficient density. While gold is mostly inert in the human body and other mammalian species, the residual gold particles shot into the skin with the gene gun leave a visible gold color region in tissues. Moreover, the consistency of skin can vary greatly from individual to individual and the amount of agent that can be deposited on the carrier particles is relatively small, presenting a major challenge for consistent and sufficient delivery of an agent.

Yet another category of pore formation techniques called magneto-poration or magnetofection use static magnetic fields to move magnetic particles into tissue and across cell membranes. As described by U.S. Pat. No. 6,853,864, US 2007/0004019, and US 2008/0006281, these techniques transport agents, including macromolecules through cell membranes and through aqueous solutions and tissues by attaching a magnetic bead to the agent and then magnetically attracting the complex of the bead and the attached biologically active molecule in a particular direction. If a sufficient number of attached macromolecules can be attracted and moved to targeted locations, e.g., through cell membranes into the cytoplasm, then a measurable biological effect can be achieved. Attachment of a magnetic bead made of a ferrous core or other magnetically active metal to an agent molecule is a complicated process, and delivery of such a particle-agent complex may leave a composite molecule within cells and tissues that may cause unwanted side effects. Another application, US 2007/0293810, called an apparatus for facilitating transdermal delivery of substances, uses a packet of electromagnetic fields and claims to effect dermal permeability for caffeine molecules using magnetic field strengths of only 3 gauss or less. This field strength is only about 5 to 10 times the Earth's magnetic field strength, which is in the 0.3 to 0.6 gauss range. Our specifications and data will show that a field strength several orders of magnitude greater is required for permeabilization of cell membranes to polypeptides and polynucleotides such as DNA, and a changing rather than static magnetic field is required. It appears that the caffeine in said patent application does not enter intracellular space, but diffuses through the interstitial space between cells of the skin. Additionally, U.S. Pat. No. 6,132,419 discusses the possibility of using an inductance device for introduction of molecules into living cells; however, it does not provide any parameters, data or reduction to practice.

The present invention improves on all of the aforementioned aspects of poration devices and methodologies by achieving efficient delivery of agents across cell membranes and by not requiring any electrodes to generate the electric field or current within the tissues, thus not requiring contact with the subject to be treated. This reduces pain and trauma from piercing the skin and deeper tissues with sharps, reduces the chance of infection from breaking the natural barrier of the stratum corneum, reduces the cost per patient and treatment by not requiring a sterile disposable electrode and simplifies the process of presently practiced electroporation, thereby promoting mass use of the technology, e.g., for mass vaccinations. The present invention is also an improvement over the use of pseudo-spark type pulses and the corona effect for achieving electroporation by not providing an ignition source for potential anesthetic or other flammable gases. The application of magnetic fields for inducing porating fields and currents within tissues without physical contact produces cell membrane permeabilization faster than the corona effect and is capable of promoting a spatially targeted effect deeper into the tissue than either the corona discharge or pseudo-spark based approach. The present invention improves over the mechanical approach of the gene gun and bead-based magnetic delivery by avoiding the complex step of attaching macromolecules onto gold beads or magnetic beads, and is different than the described magnetoporation or magnetofection, in that movement of macromolecules is not caused by magnetic attraction, but rather by strong magnetic pulses that, we propose, induce eddy currents (movement of electrons and ions) inside the targeted tissues which result in electric fields and membrane pore formation. A muscle twitch is generated upon application of the magnetic pulses described in this invention, indicating induction of electric current in the target tissue. However, as compared to muscle twitches generated by electroporation with traditional penetrating needles, the muscle movement is uninhibited and does not endanger the twitching tissue. Movement of skin or muscle tissue while it contains embedded sharps causes tissue tearing and may result in additional trauma and pain over and above the initial insertion of the sharps.

Magnetic pulses and fields have been used for other medical diagnostic or health-related applications, though few have documented a direct therapeutic effect. Examples of such magnetic devices are the nuclear magnetic resonance (NMR) or magnetic resonance imaging (MRI) devices and transcranial magnetic stimulation (TMS) devices. TMS has been used to diagnose nerve conduction abnormalities, to map the motor cortex in the brain and study sensory and cognitive deficits. More recently, the US Food and Drug Administration has cleared the use of a TMS device by Neuronetics, Inc., for use in the treatment of depression, making it one of the first therapeutic applications of magnetic fields. Compared to the understanding and applications of electric fields in living systems, there is a relative void in the understanding and uses of magnetic fields in living systems. The present invention provides for, and teaches a new paradigm in the technique of electroporation that is based on the application of magnetic field pulses with rapid changes in the magnitude and/or direction of the magnetic field, and affords a novel method for delivering biologically active agents, including macromolecules, into cells of living tissue, which may have major applications in scientific research, industrial production and in the treatment and prophylaxis of diseases.

### SUMMARY OF THE INVENTION

Michael Faraday (1791-1867) developed the concept that a change in magnetic field can be used to induce current, using the formula:

|ε|= [symbol] /dt

where |ε| is the magnitude of the electromotive force in volts, which can be expressed as an electric field by measuring it across a unit of distance in volts/cm. The vector [symbol] is the magnetic field, and t is time. By substituting in Ohm's Law, V=iR, we have:

|i|= [symbol] /dt·1/R

where i is current (concentrating here on magnitude and not direction), the vector [symbol] is the magnetic field, t is time, and R is resistance. This formula indicates that a change in the magnitude or the direction of a magnetic field induces current, and furthermore, this induced current is proportional to the rate of change of the magnitude and/or direction of the magnetic field and is inversely proportional to the electrical resistance in the medium, i.e., in this case the resistance of biological tissue. Therefore, we can directly induce currents in biological tissue without generating an electric field between electrodes, as has been heretofore done when performing traditional electroporation, e.g., with needles or microneedles placed into or onto the target tissue. Achieving electroporation by applying changing magnetic fields without using electrodes is a new paradigm. Using changing magnetic fields for transferring agents into cells is also distinct from “magnetoporation” or “magnetofection” discussed above, which rely on magnetic beads coupled to agent molecules to be moved by traditional magnetic attraction. That prior art requires much lower magnetic flux densities than the present invention, and employs an essentially constant magnetic flux, whereas the present invention requires a changing magnetic flux, magnitudes and parameters of which will be described later in these specifications. Though not fully understood, the mechanism of electroporation is postulated to involve disruption of the orderly structure of the lipid bi-layer of the cell membrane, which results in temporary openings that allow fluids, ions and molecules, including macromolecules, for which the membrane is not normally permeable to pass through. The electric fields typically used for electroporation of mammalian cells with traditional electrodes are in the range of several to hundreds of volts per centimeter (V/cm). Instead of an electric field generated with electrodes and a voltage source, this invention uses a changing magnetic field to directly induce electrical and/or ionic currents and associated electric fields which are believed to facilitate the formation of membrane pores. Once formed, these pores behave as mentioned above to permit passage of bioactive agents including polynucleotides to pass through and, in the case of DNA, transfect the cell.

Since electromotive force from electric fields generated between electrodes is not used in this invention, the traditional units of measurement of “volts per distance” to effect electropore formation is not applicable and not even calculable. Instead, mathematical expressions of magnetic flux density change (dB) per unit of time are useful for describing the effect of changing magnetic fields on membrane poration. The greater the value of dB per unit of time, the greater the magneto-motive force (as we might call the equivalent of electromotive force generated with electrodes) that directly or indirectly may cause membrane pore formation. The magneto-motive force also induces movement of charged species in the conductive aqueous environment of the biological tissue. The resultant current of charged species can be expressed as current density, in units of amps per unit of area, such as A/cm². Likewise, as described in a prior patent application by Kardos and others in 61/011,772 (2008) and PCT/US2009/000273 on variable current density single needle electroporation, the expression of current density at a given resistance value correlates to a certain extent with electroporation efficiency. In electrode configurations that result in non-linear or dissipating electromotive force with distance, the electric field strength expressed in V/cm varies at every point in space, but the approximate corresponding current density is more readily measurable than the electric field strength (V/cm). Since current and current density induced by a changing magnetic field can be readily calculated and measured (as opposed to voltage and electrical field strength) induced current density can be used as an approximate measure of poration efficiency to be expected at a certain magnetic change-of flux rate.

Electromagnetism is defined along a continuum of frequencies, of which a slice is directly perceptible for human beings in the visible range from about 400 nanometers (blue) to 700 nanometers (red), corresponding to about 10¹⁴ Hertz to 10¹⁵ Hertz, per the formula:

f=c/λ

where f is frequency, λ is wavelength, and c is the speed of light, about 3×10⁸ m/sec. The frequencies of the electromagnetic (EM) spectrum are generally divided into ELF, VLF, LF, HF, UHF. The region of interest in the present invention is the ELF, or ultra low frequency range, defined as being above 0 and up to 3 KHz, below radio frequencies. Because of the quasistatic nature of EM fields at these low frequencies, electric and magnetic fields act independently of one another, and are measured separately as either in volts (V) or tesla (T). Very low magnetic fields are measured in gauss (G), with 1 tesla=10,000 gauss, where 1 gauss is approximately equal to the Earths magnetic field strength. Furthermore, since the present invention relies on a non-static magnetic field above the frequency of 0 Hz, the magnitude or direction of which changes with time, the parameters of this invention are related to the rate of change of magnetic flux and thus measured in terms of tesla per second. In the following paragraphs and sections different embodiments of the invention as it relates to the induction of membrane poration by changing magnetic fields will be presented. To quantitatively compare the different embodiments in terms of the effect of changing magnetic fields on membrane poration, we will use standard units whose values can be correlated, in approximation, with membrane pore formation efficiency. Where units of tesla are referenced, they represent a momentary value of magnetic field strength in time, such as a maximum. Rate of change of the magnetic flux is given in units of tesla per second. In addition, since this invention claims an electroporative effect due to a changing magnetic field that induces currents within the target tissue, current density in Amps/cm² may also be referenced. The following paragraphs relate to three different embodiments of the invention that differ in relation to relative position and movement between the magnetic field(s) and the subject or tissue in which membrane poration is to be achieved.

A first embodiment is characterized by a stationary subject or tissue placed in close proximity to a stationary electromagnet that generates magnetic field pulses. See also, for example, FIG. 10. Parameters of stationary electromagnet operation with pulsed fields are in the range of 0.1-100 tesla achieved within 0.01 to 100 microseconds, single cycle and multiple cycles, monophasic and biphasic, with a pulse period of 0.01 to 10 milliseconds. This results in a magnetic slew rate (rate-of-change-of-flux) of 0.001 tesla/microsecond to 10,000 tesla/microsecond.

A second embodiment is characterized by a stationary electromagnet and a subject or tissue that is moved through the static field generated by said magnet. See also FIG. 11. Parameters of stationary electromagnet operation with target moving through the static field: Static field strength 1-20 tesla, with rotating or linearly moving subject through the field at a speed of 10 to 100 km/hour. The magnetic slew rate depends on the spatial distribution of the magnetic field. In the case where the field develops from <10% to 100% of the full strength in 10 cm, the lowest slew rate is approx. 9 tesla·(1 km/hr)/10 cm=90000 tesla/hr=25 tesla/sec, and the highest slew rate is approx. 18 tesla·(100 km/hr)/10 cm=5000 tesla/sec, or 0.005 tesla/microsecond. This method therefore is less capable of induction than the previous, and may be more difficult to implement.

A third embodiment is characterized by permanent magnets that move relative to a stationary subject or tissue. See also FIG. 12c. The parameters of moving permanent magnets (such as neodymium iron boron magnets): Permanent magnet strength 0.1 to 15 tesla rotated at 100 to 10000 RPM, to achieve a relative speed of the magnet surface to the subject of 1 to 400 km/hour. The arm radius from the center of rotation to the magnet surface is 0.01 to 1.0 meter, with the speed given by the formula: Speed=2(pi)·(radius in meters)·(RPM). The range of flux rates may be calculated as follows. With an apparatus that uses one rotating magnet, one quarter of a revolution results in a 0 to 100% change in incident magnetic flux. At 10000 RPM, a quarter turn is achieved in 1/40000 minutes, or 0.0015 seconds. With a 15 tesla magnet, the maximum rate-of-change-of-flux is therefore 15/0.0015=10000 tesla/sec or 0.01 tesla/microsecond. As evident from these calculations, the first method of the three given above yields the greatest rate-of-change of magnetic field and is therefore preferred over the other two.

There are two major steps to drug delivery by magnetopermeabilization. The first is the initial placement of the agent solution into the interstitial space of the targeted tissue, whether it is intramuscular, intradermal, subdermal, intra-organ or intra-tumoral. The second is the movement of drug molecules from the interstitial space surrounding the cells through the permeabilized cell membranes into the cytoplasm and nucleus. Therefore, in addition to facilitating the delivery of agents across cell membranes by magnetic permeabilization, another aspect of this invention describes methods of delivering the drug or DNA solution into the desired tissue in a way that is optimal in conjunction with magnetic permeabilization, or magnetopermeabilization. These two steps together are both important to effectively deliver the agent in question and, in the case of medical application, for effective treatment. A major aspect of magnetopermeabilization is the width and breadth of the region through which the dB/dt induces eddy currents, i.e., multidirectional currents, which are distinct from unidirectional currents flowing between two electrodes, as is the case in classical electroporation. A concomitant or associated method that delivers the agent solution must therefore aim to deliver the agent in an area or region that overlaps with the area or region of effective magnetopermeabilization, rather than in a concentrated “bolus” as done in the prior art. The magnetic effect is dispersed yet greatest near the region closest to the magnet; therefore these concomitant or associated ways pre-deliver the agent solution in a dispersed way with a large proportion near the targeted tissue, where the magnetic effect is greatest. Three associated ways of agent delivery are described as follows.

The first associated way of delivering the agent solution is through an improved jet injector that triangulates multiple streams of pressurized agent solution that intersect and collide near the surface of the tissue such as in the intradermal or subdermal region and lose momentum to remain near the region of collision. It is known to those practiced in the art that jet injectors tend to deliver a stream of solution under pressure deep into subdermal tissues, rather than into the dermis. Attempts at modifying injectors to deliver agent solution into the dermis have yielded inconsistent results. The improvement described herein, e.g., to provide multiple intersecting streams overcomes this inconsistency and allows more control over agent delivery into the dermis. This concept is based on the observation of intersecting or colliding water jets which have a demonstrated effect of reducing the forward momentum of each jet.

The second associated way of delivering agent solution designed for magnetopermeabilization is through an improved jet injector which provides multiple simultaneously delivering nozzles that are each smaller than the single nozzle in the prior art but operate in a concerted and near-parallel fashion to distribute the agent solution into the target tissue, e.g., in the intradermal or shallow subdermal region.

The third associated way of delivering the drug solution designed for magnetopermeabilization is by raster scanning the output stream of a jet injector (analogous to the way a television image is scanned or projected onto the surface of a cathode ray tube) which distributes the agent solution from a single nozzle across a wide surface at a shallower depth than a similar fixed nozzle of the prior art, due to the added feature of substantially always moving the position of the jet rather than allowing it to remain relatively stationary in its aim relative to the targeted tissue.

The time sequence of agent delivery and magnetopermeabilization pulses is also critical for effective membrane poration. It is well known to those practiced in the art that delivery of an agent such as a solution containing DNA must be delivered to the target tissue prior to electroporation. Even if DNA solution is delivered seconds before electroporation, efficient transfection will occur, whereas, if the electroporation pulses are applied just seconds before the delivery of DNA solution, electroporation will essentially have no effect on the efficiency of transfection. It has been postulated that this sequence is critical because electroporation produces an electrophoretic effect that drives polar molecules such as DNA in a particular direction into and through cell membranes, and this of course cannot occur if the agent is not present during the electroporation pulse. It is also well known to those practiced in the art that electroporation pulses can be delivered well after drug delivery, which allows for redistribution of the fluid and agent within the target tissue. However, long delays may allow degradation of some agents such as those containing proteins and DNA by proteases and nucleases, respectively.

### BRIEF DESCRIPTION OF THE DRAWINGS

FIG. 1 is an illustration that shows the exploded isometric view of the magnetic applicator with components identified: 101 is the electromagnet coil with cover, 102 is the central hole of the magnetic coil, 103 is the fluid receptacle for drug or agent, 104 is the injection plunger, 105 is the injector power head (operated magnetically or by compressed gas), and 106 is the cable connection for the magnetic coil.

FIG. 2 is an illustration that shows an isometric view of the interior of the 105 agent injector power head, showing a 108 second magnetic coil, with 107 ferrous metal plunger, which is connected to 104 agent injection plunger that moves as shown by arrow when the magnetic injector coil is energized.

FIG. 3 is an illustration that shows an exploded view in a shaded solid image of the major components of the magnetic applicator handle (same as in FIG. 1).

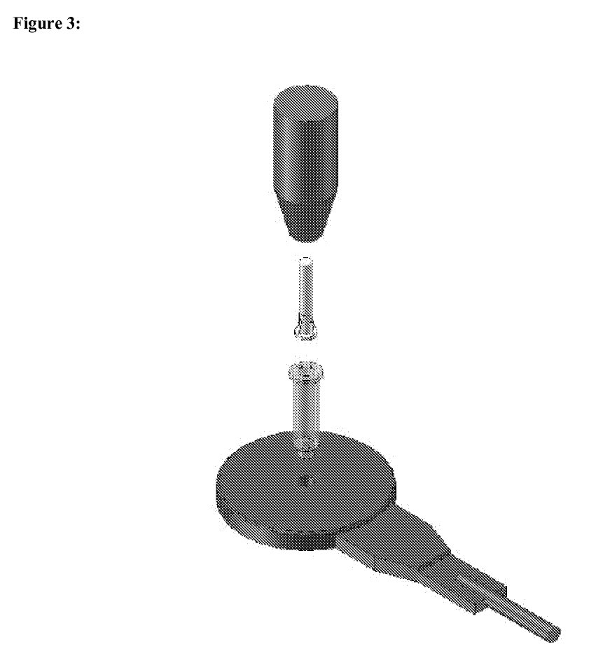

FIG. 4 is an illustration that shows an isometric view in a shaded solid image of the magnetic applicator handle assembly with agent injector mechanism attached.

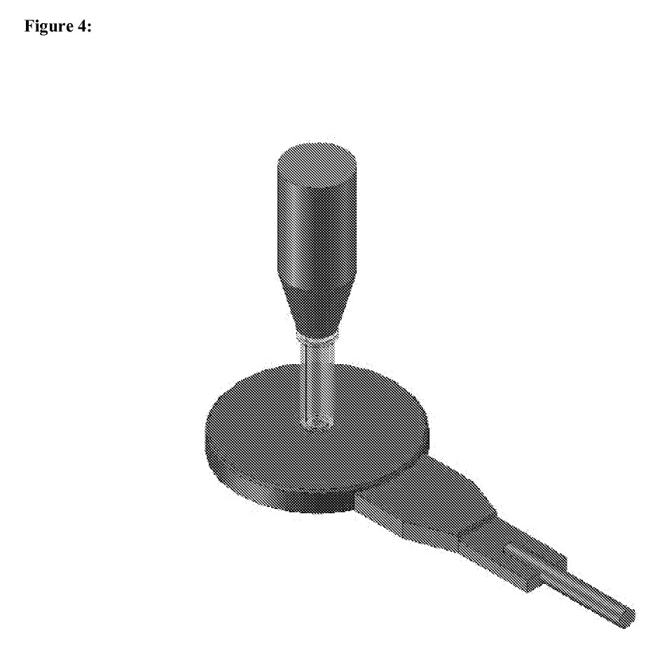

FIG. 5 is an illustration that shows the three views of the magnetic applicator, with FIG. 5a being top view, FIG. 5b being side view, and FIG. 5c being the end view, with the cable connection to the back (not visible).

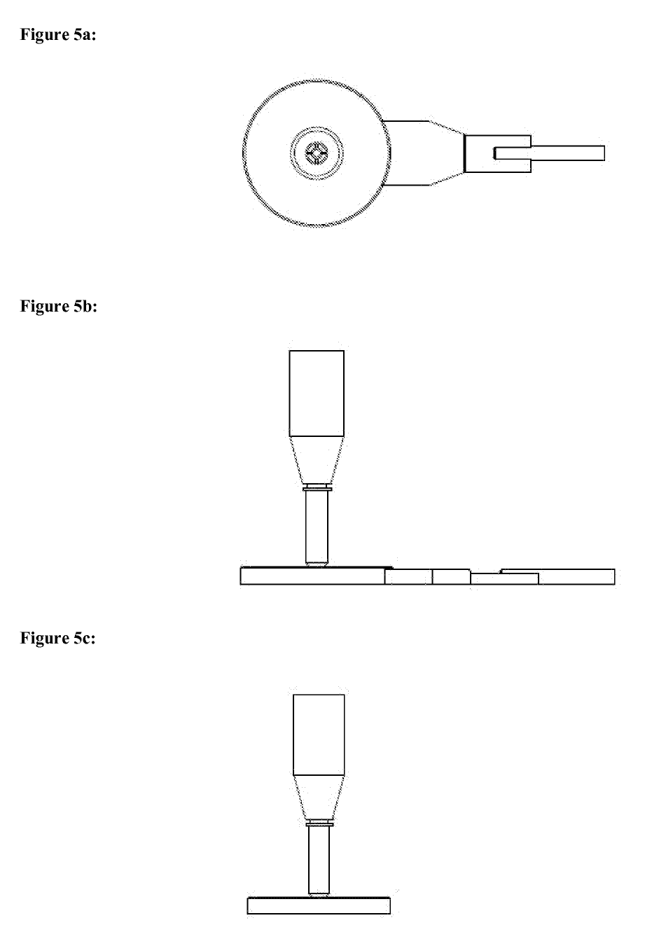

FIG. 6 is an illustration that shows the magnetic applicator assembly with 101 magnetic coil, together with the 106 connecting cable and 109 electronic power supply and electronic control assembly.

FIG. 7 is an illustration that shows 101 magnetic applicator with 102 central hole, 103 agent injector with 110 agent within injector receptacle, 111 multiple convergent aiming spouts emitting 112 converging jet streams of agent, and 113 accumulated agent at a predefined depth within tissue below injector, where the convergent jet streams meet.

FIG. 8 is an illustration that shows an alternate embodiment of the injector within the 101 magnetic coil of the applicator, with a 114 array of multiple separate jet spouts aiming a 115 substantially parallel array of multiple nozzles generating multiple agent solution streams, and 113 agent accumulated over a different geometric shape than shown in FIG. 7.

FIG. 9 is an illustration that shows the 101 magnetic applicator coil together with 116 magnetic lines of force (dotted lines) emanating in a toroidal fashion, with maximum field strength beneath the magnet, colocalized with the region 113 containing accumulated agent.

FIG. 10 is an illustration that shows the 101 magnetic coil positioned upon the 117 skin surface with 118 subcutaneous tissue, with 116 magnetic lines of force at maximum intensity below the center of the magnetic coil, where the high rate of change of the magnetic field induces 119 electroporative eddy currents within the target tissue, colocalized with the 113 accumulated agent solution that was previously injected.

FIG. 11 is an illustration that shows an alternate embodiment of the invention where the magnetic field magnitude and direction is constant, such as that in a 120 traditional MRI Magnetic Resonance Imaging toroidal magnet, and the 121 subject is swept through the magnetic field in a 122 rotating fashion, causing an incident rate of change of the magnetic field magnitude and direction on the subject's tissues, inducing electroporative currents.

FIG. 12 is an illustration that shows an alternate embodiment where an array of the 123 strong neodymium permanent magnets of typically 1 tesla strength are rotated rapidly (as indicated in FIG. 12a). FIG. 12b also shows 116 magnetic lines of force (dotted lines) emanating in a toroidal fashion, with maximum field strength beneath the magnet, colocalized with the region 113 containing accumulated agent under 117 skin within the 118 subcutaneous tissue, whereby the rotating magnetic field induces 119 eddy currents within the tissue which cause poration of cell membranes. FIG. 12c illustrates an alternate embodiment of the rotating magnet using a wheel of magnets with alternating N-S and S-N polarity orientation, which when rotated result in an incident rapidly changing magnetic flux in and below the 117 skin, next to which it is rotated.

FIG. 13 is an illustration that shows an example of the timing of the magnetic field applied by the previously described 101 magnetic coil (not shown in this diagram), obtained with an oscilloscope whose screen displays 124 trace that is representative of the magnetic field strength, where the said field rises to a maximum 125 of, for example, 4 tesla within a time 126 of, for example, less than 10 microseconds, before the illustrated monophasic pulse decays. FIG. 13b is obtained from an actual experiment.

FIG. 14 is an illustration that shows an example of the magnetic field over time similar to that shown in FIG. 13, with the difference that this pulse is a biphasic pulse, where the 127 trace displayed on the oscilloscope screen is representative of the magnetic field strength that rises to a maximum 128 of, for example, 4 tesla within a time 129 of, for example, less than 10 microseconds, before the field alternates to an opposite polarity and then decays. FIGS. 14a, 14b and 14c show variations of biphasic pulses, with FIG. 14c obtained from an actual experiment. FIG. 14d shows an expanded time base, indicating an actual rise time of approx. 400 nanoseconds, or 0.4 microseconds.

FIG. 15 shows the results from an experiment employing a reporter gene, such as a DNA plasmid coding for a fluorescent protein, which expresses a specific color (e.g., green) of fluorescence when living cells are successfully transfected with this gene. FIG. 15a illustrates marked regions 130 of skin which were injected with the reporter gene. FIG. 15b illustrates such regions of skin that have been injected with the reporter gene without subsequent poration, showing very little or no expression 131 (negative control). FIG. 15c illustrates such regions where monophasic or biphasic magnetic pulses have been applied after injection, with gene expression 132 visible in most injected regions. FIG. 15d illustrates a similar case as in FIG. 15b, except that traditional electroporation has been applied (instead of magnetic pulses) using electrodes that generate an electric field that imparts current into the injection regions, which also caused gene expression 133 (positive control).

### DESCRIPTION OF THE EMBODIMENTS

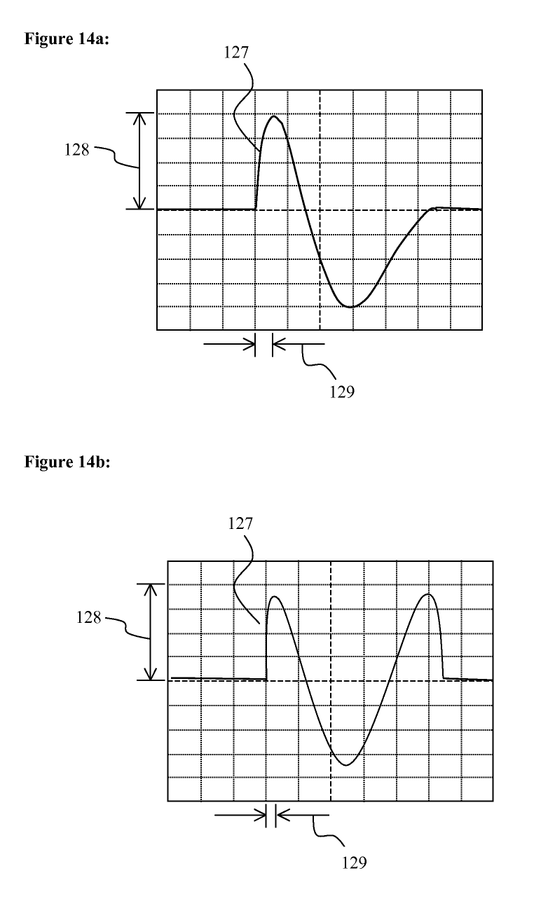

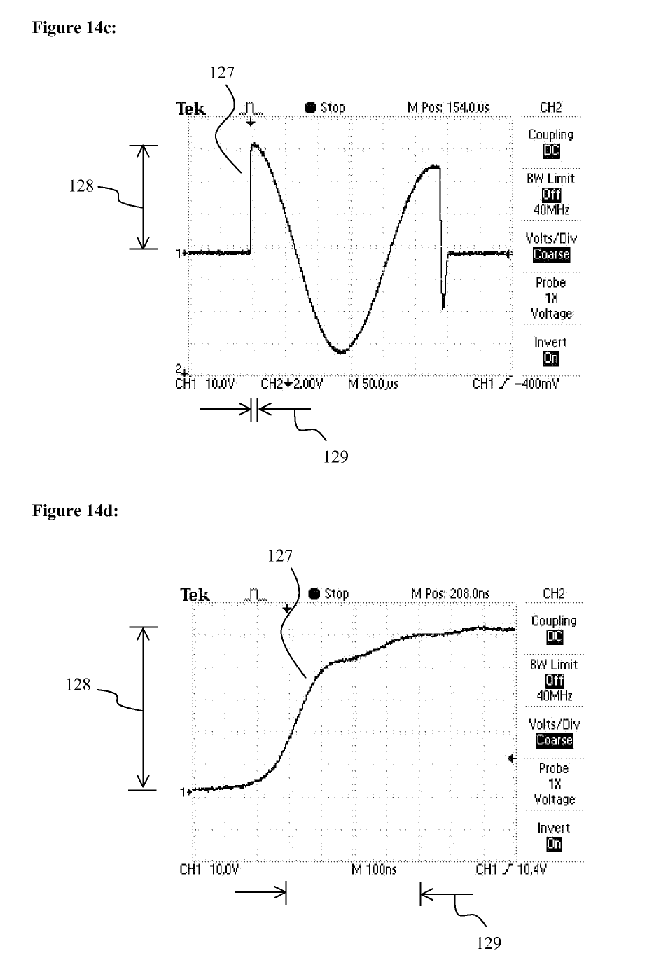

The basic embodiment, utilizes a magnetic coil with a hole through the center. The first step in the delivery of an agent, e.g., a polynucleotide (DNA, RNA in various conformations, e.g., plasmid), polypeptide (protein) or other pharmaceutical agent is a traditional intradermal, subdermal, intramuscular or intratumoral injection by needle and syringe as known by one practiced in the art of medical injections. This delivers the agent to the targeted interstitial space surrounding the targeted cells. The second step is the placement of the magnetic coil 101 over the injection site, with the injection puncture located within a circle defined by the central hole on the magnetic coil. More specifically, the center of the injected agent lies within a distance between zero and 1 cm from the centerline of the magnetic coil. The third step is activation of the magnetic coil with a fast-rising substantially DC pulse or sequential pulses of uniform or alternating polarity to produce a very large rate-of-change of magnetic flux, such as described in FIGS. 13 and 14. The method, devices and parameters of magnetopermeabilization and the effect of the magnetic pulse(s) are detailed in the following paragraphs.

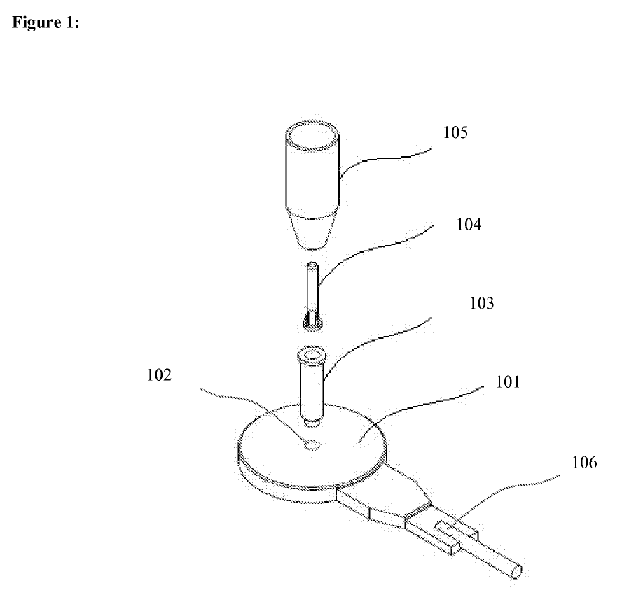

The preferred embodiment of the invention is described in FIG. 1. There is a magnetic coil (101) with a hole at the center (102), within which is placed an agent receptacle (103) containing the agent, such as DNA plasmid which includes a polynucleotide sequence that encodes for a polypeptide (protein) that is desired to be expressed by the patient's cells. To deliver the agent into a region of the subject's tissues, said agent in receptacle (103) is compressed by injection plunger (104), which is forced into said agent receptacle by an injector power head (105). The force for said injector power head is either magnetic or from compressed gas. In one embodiment, a traditional Carbon Dioxide cartridge is used to force the said plunger (104) down using the expansion of the said compressed gas. This method is known to those practiced in the art of jet injection of agent, however its use has not been described in combination with use of a rapidly changing magnetic field to porate the membranes of cells within the target tissue where the injection is delivered.

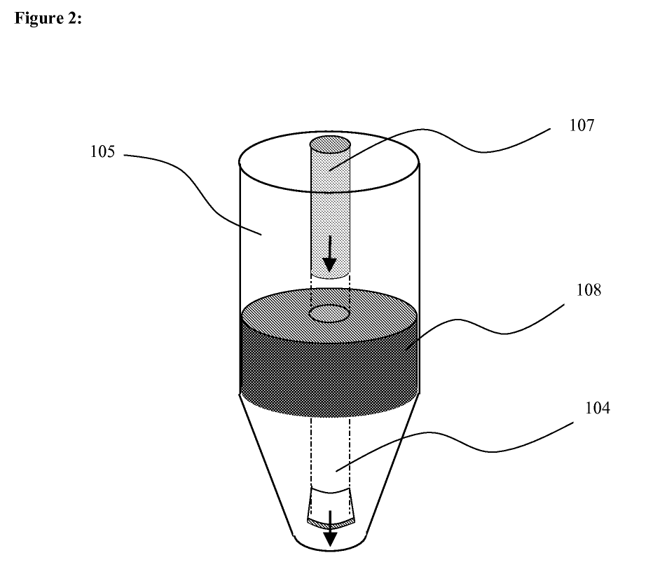

In the preferred embodiment of the injector head shown in FIG. 2, the injector power head (105) is magnetically activated with a second magnetic coil (108), which attracts a ferrous core plunger (107) in the direction shown by the arrow on part (107), and pulls said ferrous core plunger into and toward the center of the magnetic coil (108). Said ferrous core plunger (108) is connected to the injection plunger (104), which is made of a non-magnetic material, and propels the injection plunger (104) as shown by the arrow on part (104) into the agent receptacle (103) shown in FIG. 1, ejecting the agent.

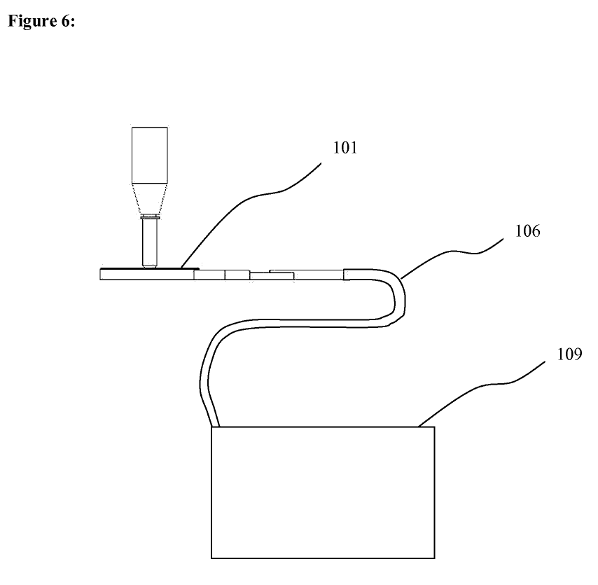

Following the injection of the agent, with a delay between zero and 1000 seconds after completion of the injection, the magnetic coil (101) is activated by its power supply and control electronics (109) through the cable (106) shown in FIG. 6 such that there is a rapid rise of magnetic flux from the background of the Earth's nominal magnetic field of approximately 1 gauss to a target of 0.1 to 100 tesla (1,000 to 1,000,000 gauss), within a brief time ranging from 1 to 1000 microseconds as illustrated in FIGS. 13 and 14. This rapid rate-of-change of magnetic flux induces sufficient eddy currents in the tissue that is co-localized with said injection site to porate the cell membranes and permit the plasmid polynucleotide or other pharmaceutically active molecule to penetrate from the interstitial extracellular space into the intracellular and intranuclear space, whereafter the known events associated with traditional electroporation-mediated drug or agent delivery, such as gene expression, follow. In the case of DNA delivery, transgene expression is designed to produce scientific or medical benefits. The sequence of said steps of 1) agent injection and 2) rapid magnetic pulse to a maximum field between 0.1 to 100 tesla within 0.01 to 10,000 microseconds, are accomplished in succession with a delay between said steps of a time period between zero and 10,000 seconds, with an optimal range between 1 and 30 seconds.

In the preferred embodiment as described above, instead of the technique of manual intradermal, subdermal, intramuscular or intratumoral injection, which requires a certain level of training and skill, the simpler and less invasive step of delivering said agent using the jet injector is performed in conjunction with the step of energizing the magnetic coil as described in the previous paragraph. A further advantage of this preferred embodiment is that co-localization of the agent and magnet placement is assured as the injector is pre-positioned within the central hole of said magnet (101).

It is most significant that said preferred embodiment provides a method by which a procedure is made available for both injection of the agent and poration of the cell membranes within the targeted tissue that avoids contact between the devices used and the subject to be treated. Thereby, this technique substantially reduces the need for sterile devices.

In one embodiment using the injector, the agent ejection actuator is powered by compressed gas such as Carbon Dioxide, Nitrogen, air, or any such compressible gas.

In the preferred embodiment using the injector, the agent ejection actuator is powered by the second magnetic coil (108). This embodiment has the advantage of not requiring a refillable or disposable compressed gas cartridge or other gas reservoir.

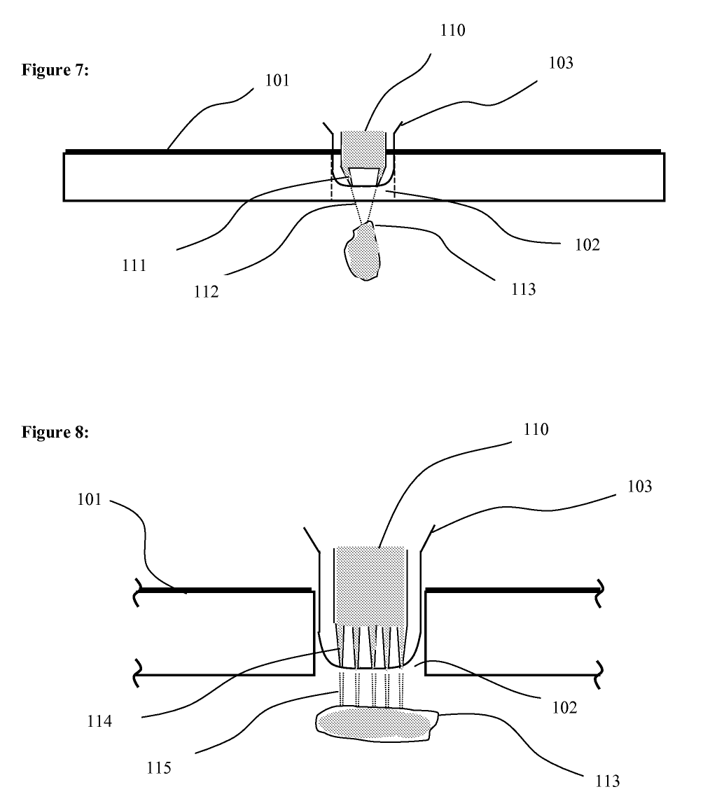

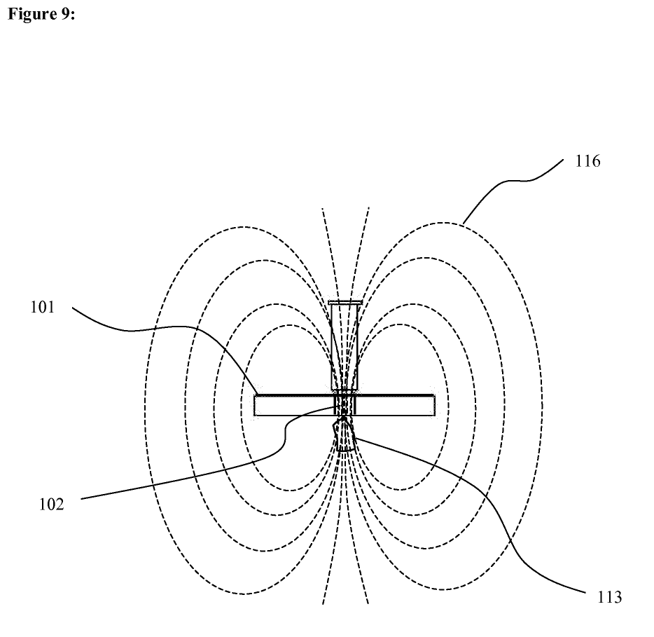

The steps of the agent delivery using either embodiment of gas-propelled or magnetically-propelled injection are diagrammed in FIGS. 7 and 8, and further described here in detail. As shown in FIG. 7, the agent receptacle (103) that contains the agent solution (110) has multiple aiming spouts (111), which eject the agent solution in convergent streams (112). At the intersection of said convergent jet streams, there is a pool of agent solution (113) starting at predefined depth below the base of the ejector. In an alternate embodiment of the aiming spouts shown in FIG. 8, an array of spouts (114) are substantially parallel and eject at a different depth and produce an agent pool (113) of different geometrical shape than that in FIG. 7. Both methods of injection result in delivery of agent substantially co-localized with the hole (102) beneath the magnet (101). The maximum magnetic flux is in the vicinity of the perimeter of said hole.

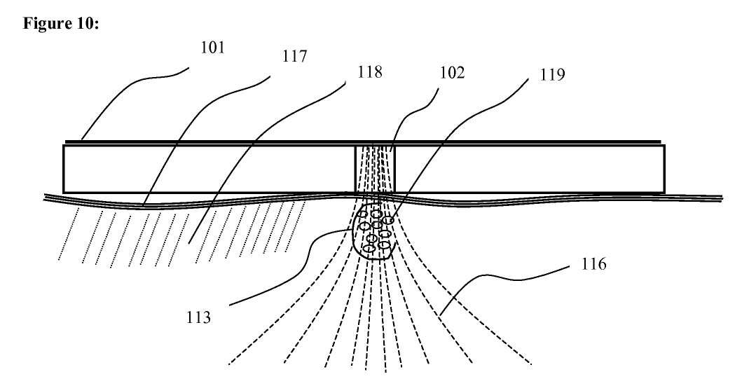

The critical method of colocalization of the delivered agent solution and the delivered magnetic field is achieved by the geometry diagrammed in FIGS. 7, 8 and 9, with FIG. 9 displaying the magnetic lines of force (116) that are at maximum intensity in the substantially same region (113) as the delivered agent solution.

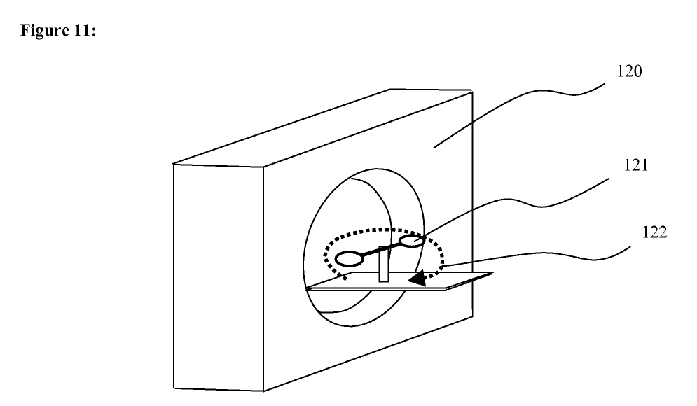

It is an object of this invention that substantially static magnetic fields, no matter how strong, are insufficient to achieve poration of cell membranes within tissue, and only rapidly changing magnetic fields are effective. The maximum intensity region described in the previous paragraph should be understood as a maximum in both space and time. The spatial distribution refers to the maximum lines of force crossing over a given region, namely the area defined by the hole (102) at the center of the magnet, and in the spatial vicinity perpendicular to the plane of the magnet, directly below the magnet hole (102) as diagrammed in FIGS. 9 and 10. The temporal distribution refers to the maximum rate of change of magnetic field [symbol] /dt, where a change in direction or change in magnitude of the field induces current. Therefore, a rapid rate of change of either direction and/or magnitude of the magnetic field to a defined maximum is an object of this patent. Both spatial and temporal changes affect the induction of the porative effect on the cell membranes. When the magnetic field changes position with respect to the target tissue, the induced current will be at right angles to both the direction of relative movement and the orientation of the magnetic field lines. When the magnetic field changes in magnitude with respect to a relatively stationary target tissue, the induced current will be in circles or eddies within the electrically conductive mass of the targeted living tissue.

In the preferred embodiment using the magnetic coil (101), where a maximum magnetic field between 0.1 tesla and 100 tesla is generated within a brief time of between 0.01 and 10,000 microseconds to achieve the peak magnetic field, there will be eddy currents (119) generated within the subcutaneous tissue (118) of the subject as shown in FIG. 10, in substantially the same region were the agent solution (113) was injected. Maximum cell membrane poration will occur in the region where the greatest change of magnetic field induces the greatest intensity of eddy currents (119).

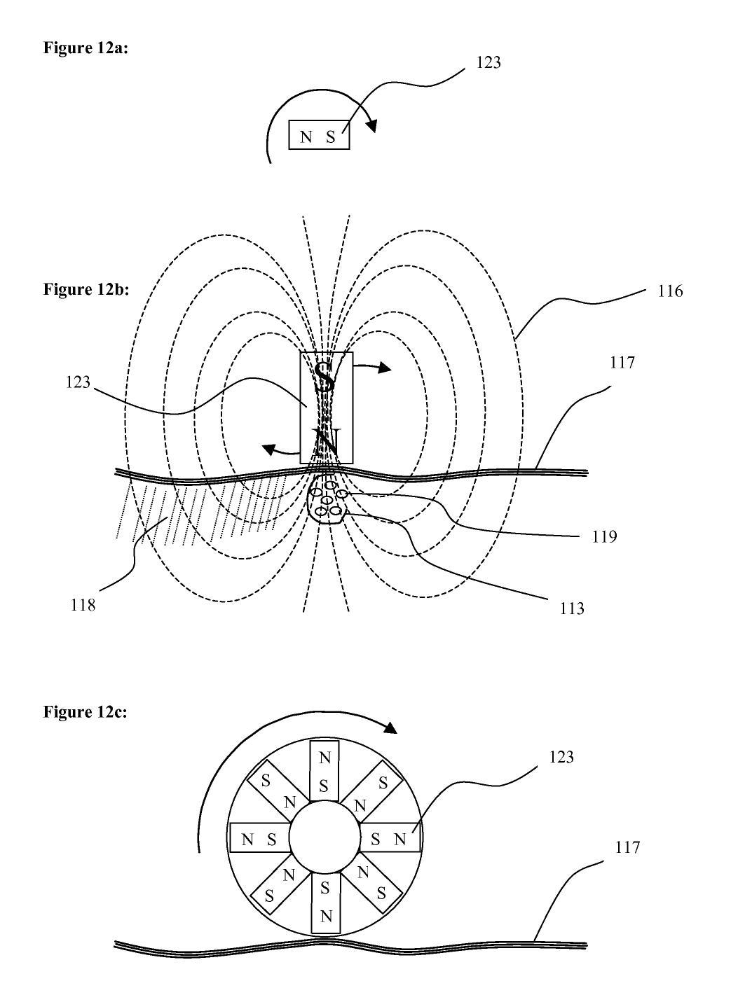

An alternate embodiment where the magnetic field magnitude remains constant but the incident direction of the magnetic field changes rapidly with respect to the target living tissue is diagrammed in FIG. 11. This configuration uses, e.g., a traditional Magnetic Resonance Imaging (MRI) type magnet (120), typically in the 1 to 10 tesla range. By having the subject living tissue in a container (121) that is rapidly rotated (122) with respect to the magnetic field, principally the direction of the field but also the magnitude is changed with respect to the subject. The greater the rotation speed, the greater is the induction of tissue currents and electroporative effect. For example, a rotation speed of 5 cycles per second where ¼ of the rotation brings the subject from crossing no magnetic lines of force to the maximum rate of crossing the lines of force, the time from zero to maximum induction would be 0.20 sec/4=0.05 seconds. If the maximum strength of the MRI magnet is 10 tesla, the rate of change of magnetic field incident on the subject would be 200 T/sec. In comparison, with the preferred embodiment, using for example a maximum 5 tesla field generated in 50 microseconds (0.00005 sec) would result in a rate of change of magnetic field of 5 tesla/5×10E-5=100,000 T/sec. Therefore, this alternate embodiment is inferior to the preferred embodiment by generating, for example, 500 times less induction and therefore a lower potential to achieve a poration effect. Furthermore, though this alternate embodiment may achieve some poration, the practicality of spinning or speeding a subject through the stationary magnetic field at high rates is impractical, due to, among other reasons, the potential complications caused by high gravitational (centrifugal) forces on the subject. This method may nevertheless be useful for animal and cell suspension applications.

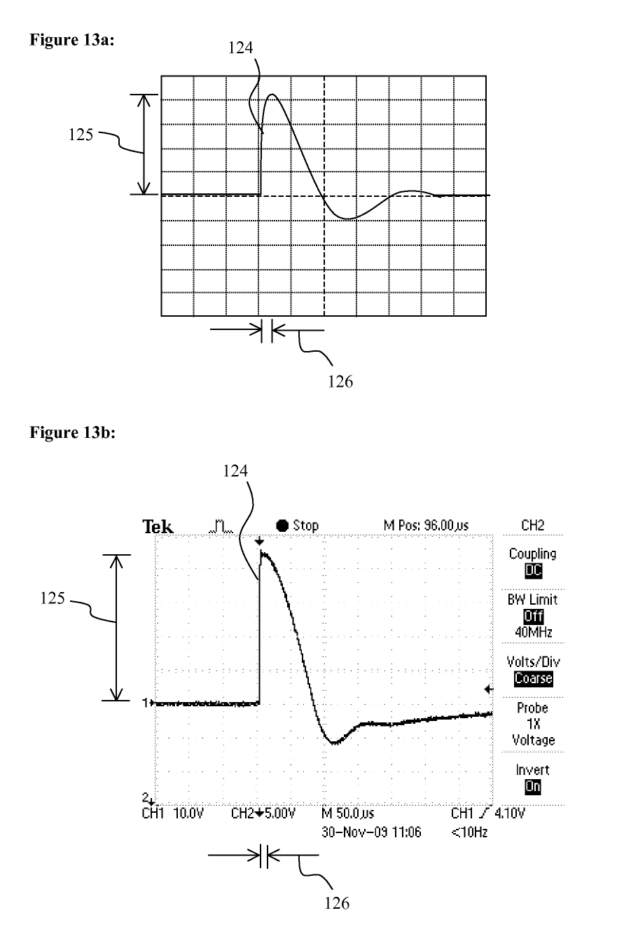

Another alternate embodiment that provides for a rapidly changing magnetic field magnitude and direction incident upon the subject is by rotating one or more strong permanent magnets as diagrammed in FIG. 12. Use of magnets such as those made with rare earth materials, e.g., neodymium iron boron or similar alloys known to those practiced in the art of making permanent magnets. Use of such magnets in the manner described herein is an object of this invention. By rapidly rotating a permanent magnetic field relative to the subject (in contrast to rotating the subject through a permanent magnetic field as in the previous alternate embodiment) induction of currents that may cause membrane poration within the tissues of a living subject may be accomplished. As diagrammed in FIG. 12b, rotating a single magnet with a single North and South pole (123) will make the lines of force (116) cross the area with injected agent (113) within the subject's tissues (118) in a way that when the North or South pole passes over the said region (113), currents (119) will be generated which will induce poration of cell membranes, which will effect transfection of the cells within the said tissue region with injected plasmid DNA gene. In this alternate embodiment, the speed of rotation of a single permanent magnet, for example, 1 tesla at 100 revolutions per second, will result in a rate of change of magnetic field of 1 tesla per ¼ turn, or 0.0025 seconds, which results in 1 T/0.0025=400 T/sec. With an improved version of this embodiment shown in FIG. 12c, which contains 8 individual permanent magnets oriented in consecutively alternating polarities, the rate of change of magnetic field can be enhanced. At the same rate of rotation as above, for example, the rate of change of magnetic field will be 1 T×8/0.0025 sec=3200 T/sec. This alternate embodiment is therefore also inferior to the preferred embodiment, since it provides approximately 30 times less induction and thus potential poration effect as the preferred embodiment with 100,000 T/sec. It is however superior to the embodiment using an MRI-type magnet in that the patient is not subjected to high gravitational forces (centrifugal forces from rotation), and does not require a large machine akin to an MRI installation. However, there is a manageable risk to the patient from a possible mechanical disintegration of the rotating mechanism, which contains heavy permanent magnets.

The preferred embodiment using a magnetic coil (101) with either a second magnetic coil (108) or compressed gas to inject the agent solution (110) achieves the best induction and porative effect, generating a field of 0.1 tesla to 100 tesla (T) in a period of 0.01 microsecond to 10,000 microsecond, thereby teaching a range for the rate-of-change of magnetic field of between 0.1 T/0.01 second=10 T/sec, to 100 T/1×10E-8 sec=10,000,000,000 T/sec or 10 GT/s (10 gigatesla per sec., or 10 kilotesla per microsecond), with a typical range of 1,000,000 T/sec to 5,000,000 T/sec (1 to 5 tesla per microsecond). The above discussion does not intend to imply a frequency or pulse-width in terms of the electromagnetic spectrum. A single pulse as defined herein is the achievement of a maximum rate-of-change of magnetic field, then the decay of that magnetic field back to zero (or the background nominal rate of 1 gauss). Use of one or multiple pulses in rapid succession are used. The range in number of pulses per application may include 1 to 10,000 at repetition rates of 0.1 per second to 100 per second (e.g. 10 pulses per second for 5 seconds, or 50 pulses per application), wherein the porative effect is realized at the initiation of each pulse when the maximum rate-of-change of magnetic field is supplied, with multiple pulses causing additive or cumulative poration.

Both a monophasic magnetic pulse, as diagrammed in FIG. 13, and a biphasic magnetic pulse, as diagrammed in FIG. 14, are objects of this invention. As indicated in the FIG. 13 example, a rise time of magnetic field magnitude (124) of 4 tesla (125) may be achieved in approx. 1 microsecond (126), which is rate-of-change of magnetic field of approx. 4 tesla/microsecond. This monophasic pulse decays and returns to a background magnetic field in approx. 200 microseconds. As indicated in the FIGS. 14c and 14d, actual rise time of the magnetic field magnitude (127) of 4 tesla (128) may be achieved in approx. 400 nanoseconds (129), which represents 4 T/4E-7 sec=10 MT/s (10 megatesla/sec, or 10 tesla/microsecond), with the biphasic pulse reversing polarity and decaying to the background magnetic field in approx. 300 microseconds. If these pulses are repeated without delay, the resultant frequency would be approx. 3000 cycles per second, or 3 KHz. This falls into the ELF or Extremely Low Frequency range on the electromagnetic spectrum. Multiple repetitions of the magnetic pulse between 1 to 3000 cycles per second are also an object of this invention; however, the typical range would be 1 cycle per minute to 100 cycles per second (0.01 to 100 Hz).

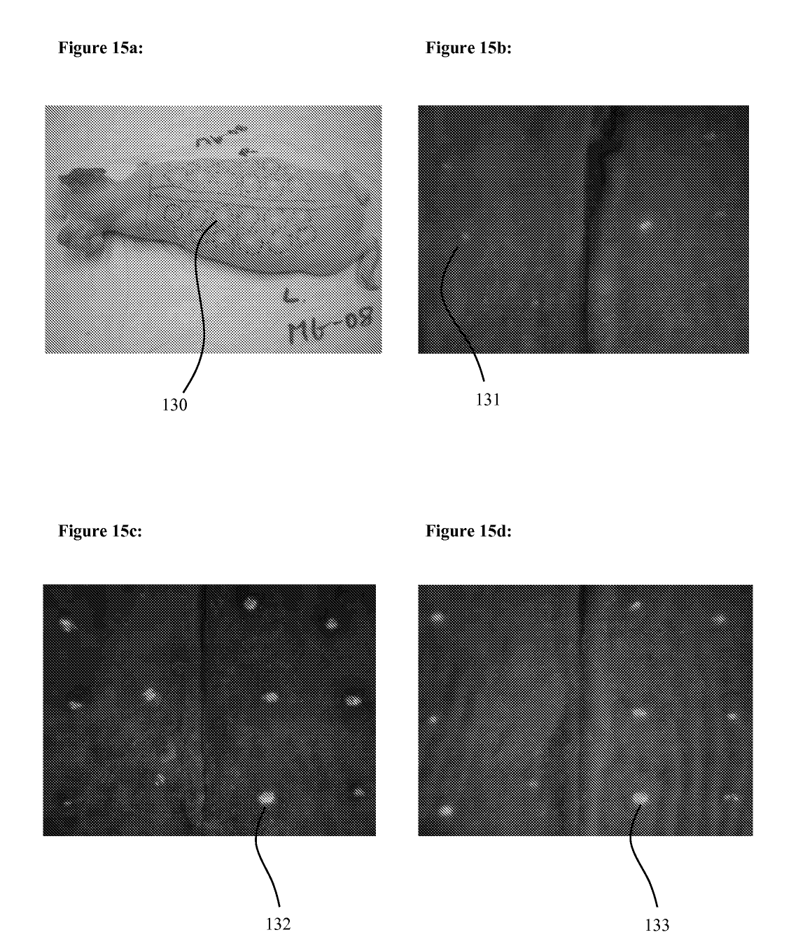

Magnetically induced electroporative effect can be demonstrated by in-vivo experiment. A typical reporter gene such as a DNA plasmid encoding a fluorescent protein can be injected in multiple places (130) on the skin of a test animal, as shown in FIG. 15a. Genes encoded by a DNA sequence and injected in a buffer solution to the skin generally do not provide substantive expression, due to the presence of nucleases, proteases and other enzymes which degrade DNA in the interstitial space between cells, where injected solutions are deposited. Plasmids or linear sequences of DNA and RNA polynucleotides must be assisted to move from the interstitial space into the endoplasmic space within cells by permeabilizing the outer cell membrane. Once inside the cell, the gene can transfect the cell by taking advantage of the biochemical machinery in the cell to express the protein encoded for by the said polynucleotide sequence in the plasmid or linear construct. Only a small amount of such transfection (131) may occur without poration as diagrammed by FIG. 15b, and this is therefore a baseline negative control. When several magnetic pulses of about 5 tesla magnitude with rise times under 10 microsecond are applied to the tissues near the injection sites within 1 minute after injection, then expression of the reporter gene such as fluorescence may be seen at the injection sites (132) after incubation, as diagrammed by FIG. 15c. Whether pulses that are monophasic (as on FIG. 15c left side) or biphasic (as seen on FIG. 15c right side) are provided to the injection sites, reporter gene expression is seen that are similar to the positive control sites (133) provided by traditional electroporation, as diagrammed by FIG. 15d, where an electric field is applied to each injection site following injection. Robust expression of the reporter gene in comparison to the controls is evidence that the non-contact method of magnetic poration of cell membranes using parameters provided in this patent may be accomplished to assist in the delivery of macromolecules such as polynucleotides which encode for genes into the cells of living subjects.

### Example 1

Experimental evidence of effective magnetopermeabilization was demonstrated by 5 sec series of monophasic pulses delivered at the rate of 10 pulses/sec for a total of 50 pulses per DNA injection site. Six injection sites were prepared, each with an intradermal injection of approximately 20 microliters of 1 mg/ml concentration gWiz-GFP (Green Fluorescent Protein plasmid from Aldevron LLC, Fargo, N. Dak.). The rise time of each magnetic pulse from zero to approximately 4 tesla was achieved within 1 microsecond, for a magnetic field rate-of-change of at least 4 tesla/microsecond. This actual pulse pattern is shown in FIG. 13b. The experimental results in terms of plasmid DNA expression for monophasic pulses are seen on the left side of FIG. 15c.

### Example 2

Similar experimental evidence of magnetopermeabilization was demonstrated by 5 sec series of biphasic pulses delivered at the rate of 10 pulses/sec, for a total of 50 pulses per DNA injection site. Six injection sites were prepared, each with an intradermal injection of approximately 20 microliters of 1 mg/ml concentration gWiz-GFP (Green Fluorescent Protein plasmid from Aldevron LLC, Fargo, N. Dak.). The rise time of each magnetic pulse from zero to approximately 4 tesla was within 1 microsecond, for a magnetic field rate-of-change of at least 4 tesla/microsecond. This actual pulse pattern is shown in FIG. 14c. An expanded time base in FIG. 14d indicates the rise time closer to 400 nanoseconds, which equates to a magnetic field rate-of-change to 4 tesla/400 ns=10 tesla/microsecond. The experimental results in terms of DNA expression for biphasic pulses are seen on the right side of FIG. 15c. When fewer pulses were delivered per DNA injection site, such as comparing 20 pulses vs. 10 pulses (2 seconds vs. 1 second at 10 pulses/sec), there was less DNA expression (data not shown). The biphasic magnetopermeabilization results are similar to the monophasic results, and both are similar to positive controls using traditional electroporation seen in FIG. 15d. Furthermore, both monophasic and biphasic results clearly show higher transgene expression than the negative controls seen in FIG. 15b, where no electric or magnetic pulses were applied.

What is claimed is:

1. A method of permeabilizing cells of a living body or live tissue characterized by temporary pores or openings within the cell membranes, whereby an extremely low frequency (ELF) magnetic field is applied to induce eddy currents within tissues encompassing said cells without physically contacting said living body which contains said tissues and cells with electrodes or other devices that conduct electric current to said tissues, wherein the magnetically induced eddy currents in said tissues increase the permeability of cell membranes to molecules, including small and large molecules, including agents, drugs, polypeptides and polynucleotides such as DNA and RNA to cross the membranes of said cells in order to increase the effect of said molecules and drugs on the said living body or live tissue, including the effect of increased transgene expression or transfection of said DNA and RNA within said living body or live tissue;

2. A method according to claim 1, wherein the said ELF magnetic field applied to said tissues and cells is provided by an electromagnetic coil whereby the direction and/or magnitude of the magnetic field relative to the position of said tissues changes with time;

3. A method according to claim 2, wherein the said electromagnetic coil is supplied with one or more pulses of current which creates one or more oscillations of magnetic field flux at the frequency of just above zero to 3 KHz (commonly referred to as the extremely low frequency or ELF range);

4. A method according to claim 3, wherein one pulse may be monophasic or biphasic;

5. A method according to claim 3, wherein the said magnetic field reaches a maximum value of between 0.1 tesla (1000 gauss) to 100 tesla (1 megagauss);

6. A method according to claim 3, wherein the said magnetic field reaches a maximum value between 0.01 microsecond and 10,000 microseconds;

7. A method according to claim 3, wherein the rate of change of said magnetic field is between 0.00001 tesla per microsecond and 10 kilotesla per microsecond;

8. A method according to claim 1, wherein the said ELF magnetic field applied to said tissues and cells is provided by one or more permanent magnets caused to move relative to said tissue whereby the direction and/or magnitude of the magnetic field relative to the position of said tissues changes with time;

9. A method according to claim 8, wherein the strength of the said permanent magnet(s) at their surface is between 0.1 tesla and 10 tesla;

10. A method according to claim 8, wherein the relative movement of the said permanent magnet(s) with respect to said tissues is between 0 and 1 kilometer per second, from rotation of an armature between 0 and 1000 revolutions per second;

11. A method according to claim 8, wherein the rate of change of said magnetic field applied to said tissue is between 0.00001 to 10 kilotesla per microsecond;

12. A method according to claim 8, wherein the said permanent magnet(s) are mounted in a manner to provide a rotating motion, for example, being mounted on a wheel whereby the permanent magnet(s) are caused to repeatedly return to the proximity of said tissue;

13. A method according to claim 1, wherein the said ELF magnetic field applied to said tissues and cells is provided by a substantially stationary electromagnet with a substantially constant magnetic field, whereby the said tissue is brought in motion with respect to the said electromagnet such that said tissue experiences a changing magnetic field direction and/or magnitude;

14. A method according to claim 12, wherein the relative motion of the said tissues in relation to the said electromagnet is between 0 and 1 kilometer per second;

15. A method according to claim 12, wherein the rate of change of said magnetic field applied to said tissue is between 0.00001 to 10 kilotesla per microsecond;

16. A method of delivering molecules comprising agents, polypeptides and polynucleotides such as DNA and RNA into cells of a living body by injecting a solution containing said molecules using pressure upon the said solution to propel said molecules into tissues containing said cells by imparting momentum to said solution, together with or followed by applying one or more pulses of an extremely low frequency (ELF) magnetic field to induce eddy currents within the vicinity of said tissue without physically contacting said living body which contains said tissue with electrodes that conduct electric current to said tissue, wherein the magnetically induced eddy currents in said tissue increase the permeability of cell membranes to the movement of said molecules from outside of said cells to the inside of said cells such as into the cytoplasm and/or nucleus of said cells in order to increase the effect of said molecules and agents on the said living tissue, including the effect of increased transgene expression or transfection by said DNA and RNA within said tissue;

17. A method according to claim 16, wherein said cells are located anywhere in the living body such as locations commonly referred to as intramuscular, intradermal, subdermal, intratumoral, intracranial and/or within any organ of said body;

18. A method according to claim 16, wherein the said pressure is generated by the use of a syringe;

19. A method according to claim 16, wherein the said pressure is generated by the use of compressed gas;

20. A method according to claim 16, wherein the said pressure is generated by the use of an electromagnet;

21. A method according to claim 20, wherein the said electromagnet is provided one or more pulses that generate a maximum magnetic field between 0.1 and 100 tesla which causes a force upon the said solution containing said molecules such as drugs to be propelled into said tissues;

22. A method according to claim 16, wherein said molecules are injected into said tissue in a particular location of said body at the same time or before the said magnetically induced eddy currents are provided to said tissue in the vicinity of said particular location;

23. A method according to claim 22, wherein the time delay between delivery of said molecules and said magnetically induced eddy currents at said particular location is between 0 and 10,000 seconds;

24. A method according to claim 16, wherein the said injecting of a solution is performed in a manner to have multiple streams of said solution;

25. A method according to claim 24, wherein said multiple streams are aimed to collide within said tissue at a particular depth;

26. A method according to claim 24, wherein said multiple streams are substantially parallel and distributed over an area between 0.1 mm² to 400 cm².

27. A method according to claim 16, wherein the said injecting of a solution is performed in a manner that moves the jet of solution to distribute said solution over an area between 0.1 mm² to 400 cm².
